(19) United States
(12) Patent Application Publication
(10) Pub. No.: US 2016/0375205 A1
(43) Pub. Date: Dec. 29, 2016

(54) Nasal Inhaler Band

(71) Applicant: Philip Wayne Cressman, Waterloo (CA)

(72) Inventor: Philip Wayne Cressman, Waterloo (CA)

(21) Appl. No.: 15/192,977

(22) Filed: Jun. 24, 2016

Related U.S. Application Data
(60) Provisional application No. 62/183,734, filed on Jun. 24, 2015.

(51) Int. Cl. A61M 15/00; A61K 9/00; A61K 31/11; A61K 31/22; A61M 11/04; A61K 31/045

(52) U.S. Cl. CPC A61M 15/0001; A61M 15/0061; A61M 11/04; A61K 31/045; A61K 31/11; A61K 31/22; A61K 9/0043

(57) ABSTRACT

A nasal inhaler band comprising a strip of air-permeable material to be worn under the nose, whereby such strip holds a vapor-releasing material which releases vapors to a user's nasal openings. Such nasal inhaler band can be worn quasi-permanently by patients, to relieve symptoms of restricted nasal patency or symptoms of air hunger (dyspnea).

### CROSS REFERENCE TO RELATED APPLICATIONS

This application is based upon and claims priority from U.S. Provisional Application Ser. No. 62/183,734, filed Jun. 24, 2015, entitled “NASAL INHALER BAND”, which is incorporated herein by reference in its entirety.

### BACKGROUND OF THE INVENTION

The present invention relates generally to devices for the introduction of vapors to nasal passages, and more specifically to devices held in front of nasal entrances to provide a flow of vapors into nasal passages during inhalation.

The inhalation of vapors (such as menthol, eucalyptol, etc.) has been used for long time in traditional and home remedy around the world for alleviation of various symptoms related to the respiratory system, such as restricted nasal patency (i.e. the sensation of restricted nasal air flow).

Traditionally, menthol or eucalyptol and/or various menthol/eucalyptol containing plants would be added into very hot water (in a bowl or other container) and the vapors coming off the surface would be inhaled. Menthol does not dissolve in water but the hot water would melt the crystals which would then float on top of the water and give off menthol vapors mixed in with water vapors generated through the application of heat. The disadvantages of this approach is that it is cumbersome, it involves hot water that could be dangerous to handle, it needlessly exposes the eyes to menthol vapors, and it only provides a short-term exposure to menthol vapors, with a significant potential for exposure to excessive concentrations of menthol vapors, known to cause irritation.

A number of menthol containing products for vapor inhalation are currently marketed to provide relief against various symptoms related to the upper respiratory system.

Various cylindrical or tube-shaped prior art devices are marketed, containing (within the tube) a substrate saturated with menthol and/or other chemicals or herbal compositions; the user is supposed to inhale through the tube, with one end of the tube inserted into a nostril. The active ingredient in such devices is typically dissolved in a liquid impregnated on a substrate, so that the air flow through and around the substrate would pick up menthol and/or other vapors. Heat is generally not applied to the substrate to assist in the generating menthol aerosol.

The disadvantages of this kind of prior art device is that they are only providing a short term exposure to an unknown and variable flow of menthol vapors; contact with the nose may also contaminate the container with bacteria or viruses.

Various other vaporizers, including hand held and table-top units, are currently marketed; they use menthol solids, oils or pads saturated with liquids, to provide menthol vapors to nasal passages. These devices work on a similar principle to the cylindrical plastic inhalers, with some added moisture or humidification; they are more convenient but are still bulky and only useful for short-term application of menthol vapors.

Various nasal strips or expanders are also marketed; such strips fit on top of the nose and are impregnated with menthol. Their main drawback is the fact that the site of vapor release is on top of the nose, thus releasing concentrated vapors very close to the user's eyes, where vapors can be irritating; their position and limited size also severely limits the intake of menthol vapors through inhalation.

Mentholated salves or creams are currently marketed that can be rubbed onto skin near nasal passages and release menthol vapors in the proximity of nasal openings. Aside from the fact that this approach can only provide low concentrations of menthol and/or other vapors in the proximity of the nasal passages, a disadvantage of this solution is that the creams can contaminate/stain hands, clothing etc.; the salves could also cause skin irritation.

Medical Plasters are also currently sold, with up to 5% menthol in a gel or paste; such plasters have been marketed for application to the skin as a treatment for bruises or strains, but not as nasal inhalers.

Vapor-releasing face masks are also presently marketed, consisting of variations of surgical or dust masks which have been modified to hold menthol solids, oils or gels to provide menthol vapors to breathing passages during inhalation. These masks were designed originally to protect the entire area around nose and mouth against dust or other particulates/irritants. The size and configuration of such masks makes them difficult to wear or to remove; such masks must also be removed every time the user needs to clear her nasal passages or to perform normal activities such as eating, drinking, or talking. When the user is resting or sleeping while wearing them, such face masks are also much less comfortable. Over time, saturation of the entire mask fabric with menthol vapors could lead to release of such vapors in the proximity of the eyes, causing eye irritation as well.

Research has established that menthol is a trigger to the “Transient Receptor Potential Cation Channel subfamily M member 8” (TRPM8) receptor (also known as the cold and menthol receptor 1, CMR1) present in the human nasal vestibules; stimulation of the TRPM8 receptors by cool air, menthol vapors (or other vapors) is known to result in an improvement in nasal patency (i.e. sensation of nasal airflow).

Stimulation of TRPM8 receptors also relieves the symptoms of “air hunger” (dyspnea), which is a subjective feeling of quasi-suffocation experienced by patients who otherwise have normal blood oxygen levels (i.e. blood oxygen levels that do not warrant supplementary oxygen treatment).

Thus, there is an unmet need in the market for a lightweight nasal inhalation device that can be worn for extended periods of time (including during sleep or while talking/eating), in which the menthol and/or other ingredients do not come into direct contact with skin, placed as far away from the eyes as practical, and which allows for control of the amount of vapor introduced over various time periods to the nasal passages during inhalation. There is a further need for such devices to be lightweight, compactly packaged for ease of storage and carry, and to offer convenient refill options.

### SUMMARY OF THE INVENTION

The present invention responds to this unmet market need by providing a device fitted on the upper lip and in front of the nasal openings of a user, releasing a flow of vapors into nasal passages during inhalation. The device can be worn for extended periods of time, including during sleep and during talking/eating. If the nose needs to be cleared, the device can simply be temporarily moved down to the chin, giving the user access to blow the nose or clean it with paper tissue.

In a preferred embodiment, the present invention consists of a band or a strip of air-permeable fabric (with one or more layers, woven or non-woven), shaped to fit above the lip and in front of entrance to nose and adapted to hold vapor-releasing solids, liquids or gels. In a preferred embodiment, the air permeable band is held in front of nasal passages (and in contact with the nose and upper lip) by elastic straps extending back from the band to wrap around the ears.

### BRIEF DESCRIPTION OF THE DRAWINGS

FIG. 1 is a side view of a preferred embodiment of the present invention in use on the face of a user, showing elastic strap around ears.

FIG. 2 is a side view of a preferred embodiment of the present invention, with an added cross-sectional view of the portion of the inhaler band that sits under the nose of the user (an A-B cross-sectional view of which is depicted at the bottom of FIG. 2).

FIG. 3 is a front view of another preferred embodiment of the present invention in use on the face of a user, showing an external vapor-releasing patch applied to the outside of the inhaler band, in the area that sits under the nose of the user.

FIG. 4 is a front view of an Adhesive Strip containing agonists (Menthol or others), which may optionally be fitted, as a refillable/replaceable menthol container, to the portion of the inhaler band that sits under the nose of the user.

### DETAILED DESCRIPTION OF A PREFERRED EMBODIMENT

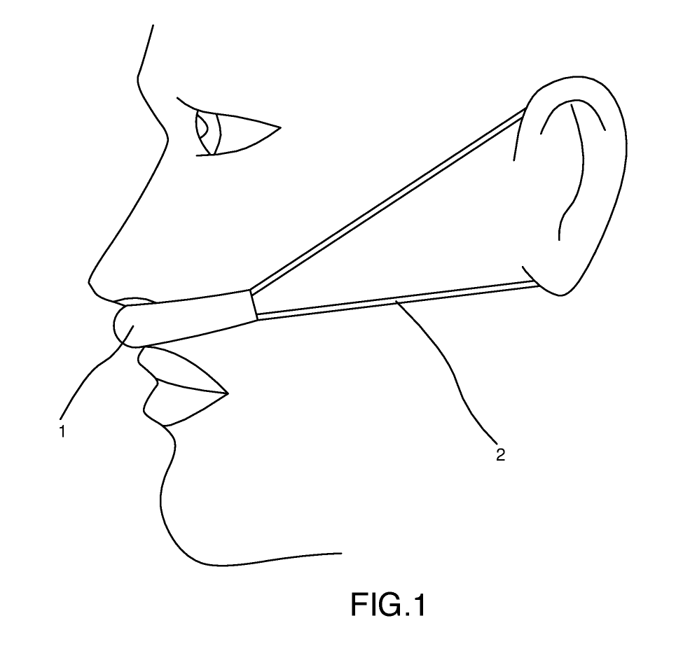

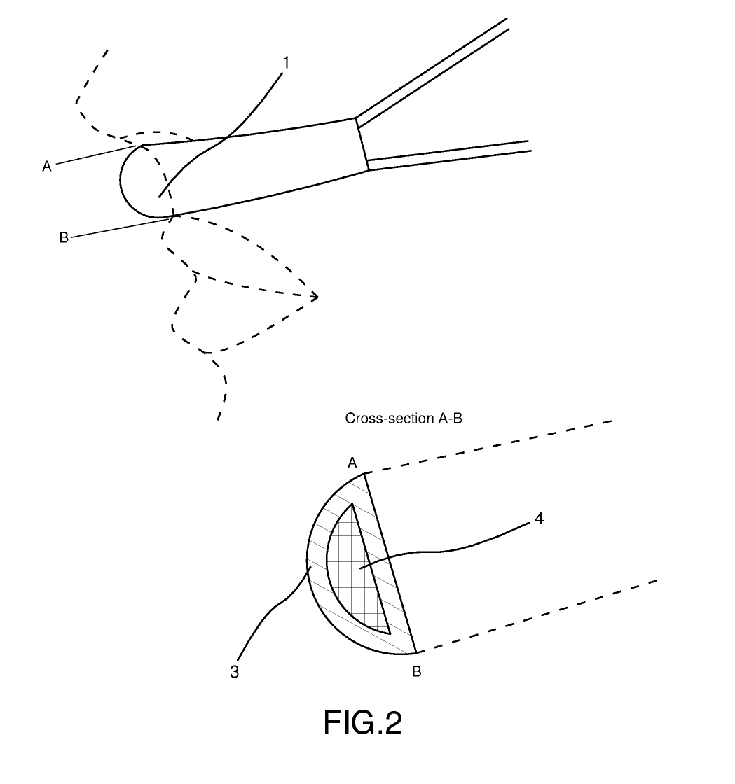

Referring now to the invention in more detail, in FIG. 1 and FIG. 2 there is shown a preferred embodiment of this invention consisting of a band (or a strip) 1 of air-permeable fabric (with one or more layers, woven or non-woven), shaped to fit above the lip and in front of entrance to nose, and adapted to hold vapor-releasing solids, liquids or gels. As shown in FIG. 1 and FIG. 2, in a preferred embodiment, the air permeable band is held in front of nasal passages (and in contact with the upper lip and proximal to the nasal entrances) by elastic straps 2 extending back from the band to wrap around the ears of the user.

As shown in the cross-section drawing in FIG. 2 (at the bottom), the inhaler band of a preferred embodiment has an air-permeable outer layer 3, containing inside an absorbent material 4, impregnated with or holding vapor-releasing material.

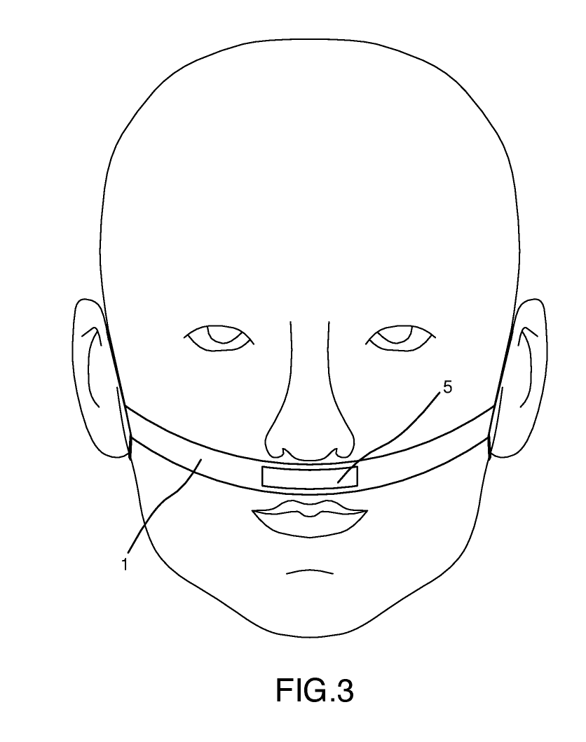

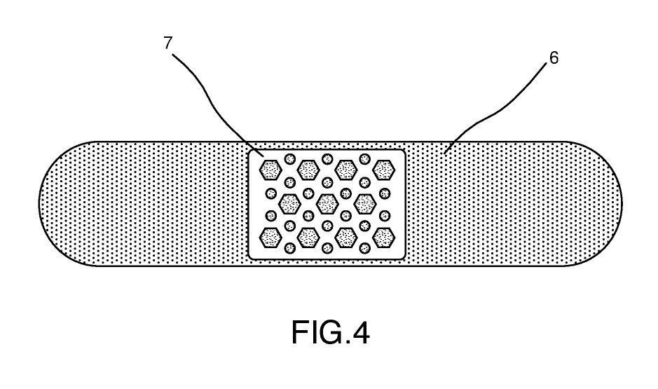

In another preferred embodiment shown in FIG. 3, the inhaler band (still held under the nose by elastic straps wrapped around the ears) may consist of at least one base layer (made of air-permeable fabric), on the outside of which a vapor-releasing patch 5 is applied. In further preferred embodiments, one or more vapor-releasing patches may be applied to the inside (skin-facing) side of the base layer, or may wrap around the base lawyer, or be placed between base layers. FIG. 4 shows a typical patch 5 which, in a preferred embodiment, consists of an adhesive strip 6, with a central absorbent pad 7; the vapor-releasing material is held within, or on the surface of, the absorbent pad 7. Replacement patches pre-filled with vapor-releasing material can be provided in sealed individual packages, whereby the user would peel off the old (depleted) patch from the base layer and replace it with a fresh patch.

In another preferred embodiment of this invention, the vapor-releasing material is inserted into the band 1 between layers of an air permeable fabric, and is permanently fixed in place during the assembly of the inhaler band.

In other embodiments, the band 1 is pre-fitted with a pouch or a pocket, and the vapor-releasing material is provided separately, sealed into an air permeable fabric container, to be inserted by the user into the pouch or pocket before use. Refills consisting of additional doses of vapor-releasing material (sealed into air permeable fabric containers) may be provided to the user to replace the fabric containers depleted of vapor-releasing material through prolonged use.

In another preferred embodiment of this invention, the vapor-releasing material is contained in an air-permeable medical plaster to be applied to the band 1 by the end user.

In another preferred embodiment of this invention, the vapor-releasing material is provided as a solution or emulsion which is to be applied to an absorbent and air-permeable portion of the band 1 by the end user.

Any vapor-emitting TRPM8 agonist ligands or mixture of agonist ligands (suitable for human use) can be used as the active ingredient in the inhaler band of this invention, either neat (crystals, powder, flakes, particles, wax, liquid, pellets, blocks, powders, gels, films, sachets, and the like) or in a carrier vehicle (suitable for storing solid or liquid TRPM8 agonist ligands and for releasing vapors of such stored TRPM8 agonist ligands), such as a solution, suspension, dispersion, emulsion gel, liquids impregnated on pads or sponges, crystals dissolved in Eucalyptus Oil or dissolved in other oils and liquids with solvent/gelling properties, and the like. Moreover, the TRPM8 agonist ligands may be releasably contained by a microcapsule, an absorbent material, a cell, an adhesive, an emollient-containing composition, a solid support, a nanophase particulate structure and the like. In a preferred embodiment of this invention, the stored active ingredient (the TRPM8 agonist ligand in its non-vapor form) is separated from the skin by at least one layer of fabric and it does not come into direct contact with skin.

According to a preferred embodiment of this invention, typical TRPM8 agonist ligands used are: menthol, AG-3-5, Cool-actP, Cooling Agent 10, FrescolatMGA, FrescolatML, geraniol, hydroxycitronellal, linalool, Methyl Lactate, PMD38, WS-3, and WS-23, and any other similar substances known to activate TRPM8.

For example, Menthol is a volatile substance; menthol crystals have a melting point of about 41-44° C., and they will sublimate even at room temperature; the rate of evaporation or sublimation increases dramatically with temperature. Vapor pressure above menthol crystals at 20 deg. C. (room temperature) is 0.083 mmHg, but when heated to 34 deg. C. (a typical temperature radiant according to this invention, achieved due to the heat from skin and exhaled air) the vapor pressure is 0.2385626 mmHg.

In a preferred embodiment of this invention, the inhaler band is fitted on a user's upper lip and in front of the nasal openings, so as to absorb heat from both the skin above the upper lip and from the warm air exhaled from the nostrils. Such absorbed heat will raise the temperature of the inhaler band and of the vapor-releasing material contained inside the inhaler band, which in turn will generate a higher release rate of vapors of menthol or of other TRPM8 agonist ligands stored therein. Assuming that the temperature of the vapor-releasing material becomes close to the human body temperature (when the inhaler band is worn according to the present invention), this could enable an approximate calculation of the vapor pressure of the TRPM8 agonist ligand at that temperature, and permits a rough adjustment of the quantity of TRPM8 agonist ligand pre-loaded inside the inhaler band at the time of manufacture, and thus enables a rough control of the amount of vapor introduced over various time periods to the nasal passages during inhalation.

The device of this invention is lightweight; the elastic loops extending around the ears (or behind the head) allow the user to temporarily move down the band (to chin level or below) when the nose needs to be blown or cleared of fluids, then the band can be placed back under the nose. The small size and profile of the band makes it comfortable to wear and does not interfere with eating, talking, resting or even sleeping.

The device of this invention can be packaged in sealed plastic bags for ease of storage and carry. Storage in an air tight plastic bag at temperatures below 35 deg. C. is expected to give a minimum of 2 year shelf life for the inhaler band herein.

While the foregoing written descriptions of preferred embodiments of this invention enable one of ordinary skill to make and use what is considered presently to be the best mode thereof, those of ordinary skill will understand and appreciate the existence of variations, combinations and equivalents of the specific embodiments, systems, methods and examples herein. Accordingly, the invention should therefore not be limited by the above described embodiments, systems, methods and examples but by all embodiments systems and methods within the scope and spirit of the invention claimed herein.

What is claimed is:

1. A nasal inhaler band comprising:
A strip of air-permeable material adapted to fit under the nasal openings and against the upper lip of a user;
Said strip held in place by one or more holding means extending from said strip;
Said strip adapted to hold a vapor-releasing material to present vapors to a user's nasal openings.

2. The nasal inhaler band of claim 1, whereby said strip consists of two or more layers of air-permeable material.

3. The nasal inhaler band of all previous claims, whereby the vapor-releasing material contains an inert material to provide bulk and softness.

4. The nasal inhaler band of all previous claims, whereby the vapor-releasing material contains one or more TRPM8 agonist ligand.

5. The nasal inhaler band of claim 4, whereby the TRPM8 agonist ligand is selected from the group consisting of:
menthol, AG-3-5, Cool-actP, Cooling Agent 10, FrescolatMGA, FrescolatML, geraniol, hydroxycitronellal, linalool, Methyl Lactate, PMD38, WS-3, and WS-23, or combinations thereof.

6. The nasal inhaler band of claims 4 and 5, whereby the TRPM8 agonist ligand is combined with other fragrant herbs or oils selected from the group consisting of: eucalyptus, cinnamon, rose, tea tree, rosemary, thyme, or combinations thereof.

7. The nasal inhaler band of all previous claims, whereby the vapor-releasing material is inserted between layers of air permeable material and permanently fixed in place during the assembly of the nasal inhaler band.

8. The nasal inhaler band of all previous claims, whereby the vapor-releasing material is provided separately, sealed into an air-permeable fabric container to be inserted by the end user into a pocket fitted into said strip.

9. The nasal inhaler band of all previous claims, whereby the vapor-releasing material is provided separately as an air-permeable medical plaster to be applied to said strip by the end user.

10. The nasal inhaler band of all previous claims, whereby the vapor-releasing material is provided separately as a solution or emulsion to be applied to said strip by the end user.

11. The nasal inhaler band of all previous claims whereby the air-permeable material is of any color and with visible printing or other graphics applied to the outside surfaces.

12. The nasal inhaler band of all previous claims whereby the holding means are selected from the group consisting of: two or more elastic straps wrapped around the user's ears; two or more inelastic straps wrapped around the user's ears;

13. The nasal inhaler band of claims 1 to 11 whereby the holding means are selected from the group consisting of: one or more elastic straps wrapped around the user's head; one or more inelastic straps wrapped around the user's head.

14. The use of the nasal inhaler band of claims 1-13 to relieve symptoms of restricted nasal patency when suffering from an upper respiratory ailment selected from the group consisting of: common cold, influenza, nasal allergic irritation, allergic rhinitis, sinus inflammation, or combinations thereof.

15. The use of the nasal inhaler band of claims 1-13 to relieve symptoms of air hunger in patients suffering from dyspnea who have blood oxygen levels that do not require supplementary oxygen treatment.
